(19) United States
(12) Patent Application Publication
(10) Pub. No.: US 2005/0261658 A1
(43) Pub. Date: Nov. 24, 2005

(54) Protective under garment

(76) Inventor: Joe Baumchen, Chicago, IL (US)

Correspondence Address: CARDINAL LAW GROUP, Suite 2000, 1603 Orrington Avenue, Evanston, IL, 60201, US

(21) Appl. No.: 11/136,691

(22) Filed: May 24, 2005

Related U.S. Application Data
(60) Provisional application No. 60/573,796, filed on May 24, 2004.

(51) Int. Cl. A61F 13/15

(52) U.S. Cl. 604/402

(57) ABSTRACT

An incontinence device includes at least one absorber flap. The absorber flap includes an absorption layer, a liquid barrier layer adjacent the absorption layer, and an attachment portion positioned at an upper region of the flap to attach to a front leg portion of an undergarment.

### RELATED APPLICATIONS

This application claims priority to U.S. Provisional Application No. 60/573,796 filed May 24, 2004, titled “Underwear” by Joe Baumchen, the entirety of which is incorporated by reference.

### FIELD OF THE INVENTION

This invention is generally directed to men's underwear. The invention is more particularly directed to a protective undergarment suitable for absorbing small amounts of leaked urine.

### BACKGROUND OF THE INVENTION

Many individuals begin to leak urine as they reach their golden years. In severe cases, they develop incontinence, which requires the use of underwear that resembles in many respects a baby diaper. Those who do not develop full incontinence, may still develop such a condition such as urge incontinence or stress incontinence, or may have less serious conditions involving leakage of urine. Even small leaks of urine can be embarrassing if the amount is enough to show on the wearer's outer clothing. These men, however, do not need full diapers and may find the use of such devices unsettling.

Other products are available to fill the gap between full incontinence and full control. These products are made to fit inside brief-style underwear. These products cause several problems. They pull the briefs away from the wearer's body, for example. Furthermore, the bulge can be unsightly and embarrassing to some users.

Accordingly, a need exists for a urine-absorbing under garment that addresses the problems of the prior art. The present invention advances the state of the art.

### SUMMARY OF THE INVENTION

The invention provides for an incontinence undergarment device. The incontinence device includes at least one absorber flap, the absorber flap including an absorption layer; a liquid barrier layer adjacent the absorption layer, and an attachment portion positioned at an upper region of the flap to attach to a front leg portion of an undergarment.

Another aspect of the invention provides an incontinence undergarment. The incontinence undergarment includes a boxer shorts, a pair of absorber flaps, each absorber flap including an absorption layer; a vapor barrier layer adjacent the absorption layer, and an attachment portion positioned at an upper region of the flap to attach to a front leg portion of the boxer shorts, wherein a portion of the absorber flaps hangs below the front leg portions when a user is standing.

Yet another aspect of the invention provides for an incontinence undergarment, including a boxer shorts and flap means for absorbing urine attached to a leg portion of the boxer shorts.

The foregoing and other features and advantages of the invention will become further apparent from the following detailed description of the presently preferred embodiments, read in conjunction with the accompanying drawings. The drawings are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 is a front view of one embodiment of an incontinent garment made in accordance with the present invention.

FIG. 2 is a side view of the incontinent garment illustrated in FIG. 1.

FIG. 3 is a front view of another embodiment of an incontinent garment made in accordance with the present invention.

FIG. 4 is a cross section of a portion of the incontinent garment illustrated in FIG. 1.

FIG. 5 is a cross section of a portion of another embodiment of an incontinent garment made in accordance with the present invention.

FIG. 6 is a front view of another embodiment of an incontinent garment made in accordance with the present invention.

FIG. 7 is a cross section of a portion of the incontinent garment illustrated in FIG. 6.

FIG. 8 is a top view of another embodiment of an incontinent garment made in accordance with the present invention.

FIG. 9 is a front view of another embodiment of an incontinent garment made in accordance with the present invention

FIG. 10 is a front view of another embodiment of an incontinent garment made in accordance with the present invention.

### DETAILED DESCRIPTION OF THE ILLUSTRATED EMBODIMENT

Throughout the various figures, like reference numbers refer to like elements.

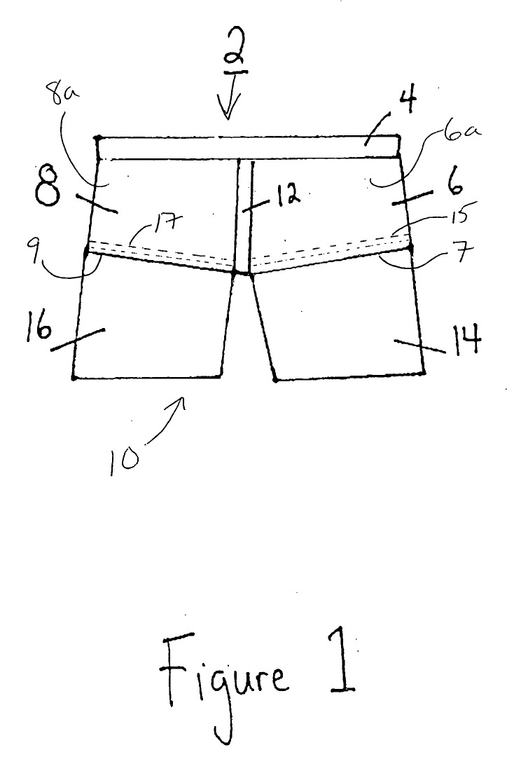

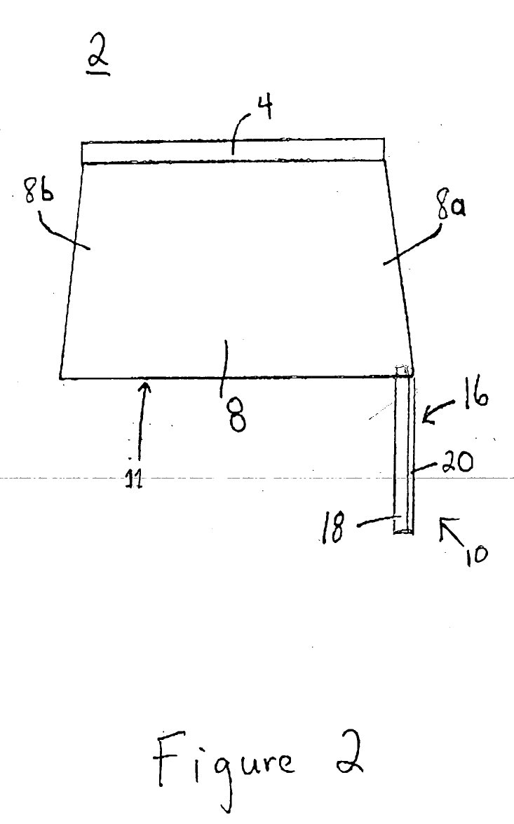

FIGS. 1 and 2 illustrate one embodiment of incontinence undergarment 10 according to the present invention. In one embodiment, incontinence undergarment 10 is operably attached to garment 2. Incontinence undergarment 10 may be removably or fixedly attached to garment 2. Garment 2 may be any conventional underwear type garment as is known in the art. The below description will be addressed to the use of the incontinence undergarment 10 with men's boxer shorts the construction and style of which are well known. Garment 2 includes a waistband 4. Waistband 4 is a circular member to hold the garment 2 on the wearer's hips. In one embodiment, waistband 4 is made of an elastic material.

Extending downward from waistband 4 are legs 6 and 8, which define openings 7 and 9 through which the wearer's legs will extend. Legs 6 and 8 have front panels 6a and 8a, and rear panels 6b and 8b. FIG. 2, illustrates a side view of the right side of the garment, showing front sides 8a and rear side 8b of leg 8.

Legs 6 and 8 join on rear sides 6b and 8b, on front sides 6a and 8a, and at crotch 11. In one embodiment, a fly 12 is located at the conjunction of front sides 6a and 8a.

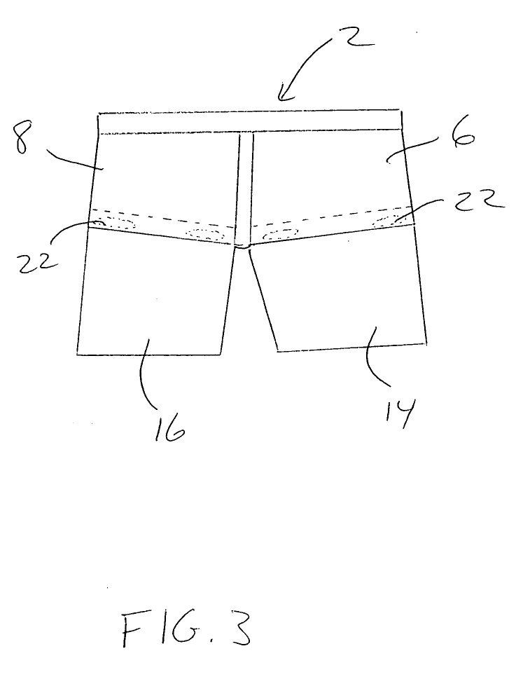

Incontinence device 10 extends downward from openings 7 and 9 of legs 6 and 8. Incontinence device 10 comprises at least one absorber flap. As illustrated, extending downward from the front sides 6a and 8a are absorber flaps 14 and 16. In one embodiment, absorber flaps 14 and 16 are designed to be approximately as wide as the thigh of a user or half the circumference of the thigh. In one embodiment, absorber flaps 14 and 16 are integral with leg walls 6 and 8. In one embodiment, an upper portion 15 and 17 of absorber flaps 14 and 16 are sewn to a lower portion of legs 6 and 8. In another embodiment, illustrated in FIG. 3, absorber flaps 14 and 16 are removably attachable to legs 6 and 8 at at least one attachment point 22. Absorber flaps 14 and 16 can join to leg 6 and 8 by snaps, hook-and-loop, zippers, buttons, or any other appropriate fastening device as are known in the art. In other embodiments, the user can attach one flap, two flaps, or more flaps to suit a particular need. In one embodiment, absorber flaps encircle the user's leg.

Absorber flaps 14 and 16 can be of a length comfortable to the user. In one embodiment, flaps 14 and 16 extend approximately one-half way down the user's thighs when the user is standing. However, shorter or longer lengths can be used. Alternatively, a series of flaps could be used so that, for example, two short flaps could be attached together by snaps, hook-and-loop, zippers, buttons, or any other appropriate fastening device to make one longer absorber flap. Absorber flaps are sized and positioned to be discreet and unnoticeable to those other than the user.

In the following discussion, reference will be made to a single absorber flap, 16. Referring to FIGS. 2 and 4, absorber flap 16 includes an inner absorption layer 18 and an impermeable outer liquid barrier layer 20. Absorption layer 18 is made of an absorbent material. In one embodiment, absorption layer 18 is cotton. In another embodiment, absorption layer 18 is made of a super absorbent polymer (SAP). In one embodiment, an absorption flap composed of a SAP that is ultra-thin to aid in providing a discreet incontinent device. In other embodiments, absorption layer 18 may be composed of materials such as, for example, cloth, paper wadding and cellulose fiber as are known in the art.

Impermeable outer liquid barrier layer 20 is a water-resistant material to help keep the user's clothing dry. Liquid barrier layer 20 is a non-porous hydrophobic substance. In one embodiment, liquid barrier layer 20 is a polyethylene film. In another embodiment, liquid barrier layer 20 comprises plastic sheeting or other waterproof material as is known in the art. Alternatively, water resistance can be achieved by impregnating the outer side of absorber flap 16 with a water-resistant coating.

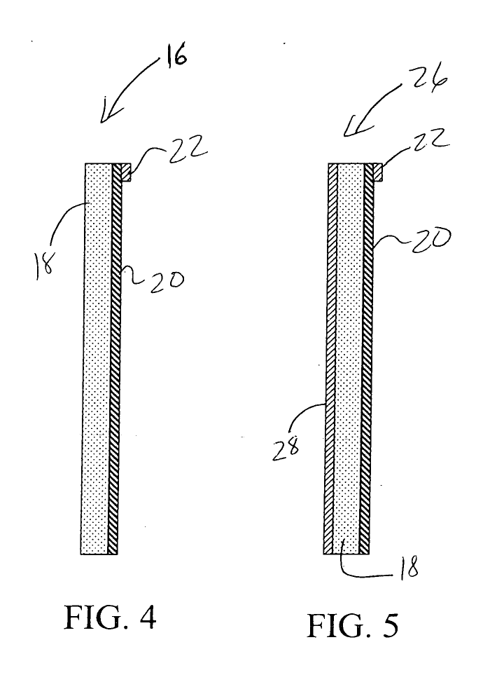

FIG. 5 illustrates another embodiment of an absorber flap 26 made in accordance with the present invention. Absorber flap 26 includes absorption layer 18 and liquid barrier layer 20. Absorption layer 18 and liquid barrier layer 20 may be the same or similar to those described above with respect to FIG. 4. Absorber flap 26 also includes a top-sheet layer 28 disposed adjacent absorption layer 18. Top-sheet layer 28 is worn adjacent or in contact with the user's skin. In one embodiment, top-sheet layer 28 is a composed of a porous hydrophobic material such as, for example, polyester or polypropylene non-woven fabric. Top-sheet layer 28 functions to allow urine to flow through it, keep liquid away from the user's skin and retain the structural integrity of absorption layer 18.

In use, if the user leaks urine, as leakage occurs, it is absorbed by absorption layer 18 of the absorber flap 16. Liquid barrier layer 20 prevents leakage through to the wearer's outer garment.

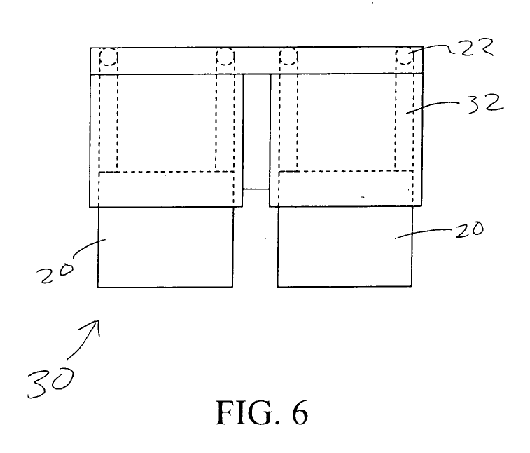

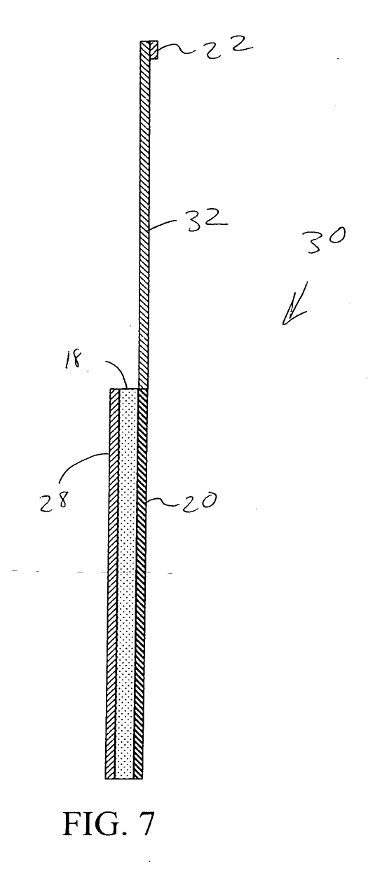

FIGS. 6 and 7 illustrate another embodiment of incontinence device 30 made in accordance with the present invention. Incontinence device 30 is similar to the triple layer incontinence device 26 illustrated in FIG. 5, including an absorption layer 18, a liquid barrier layer 20 and a top-sheet layer 28. Incontinence device 30 also includes an elongated attachment portion 32 for removable attaching incontinence device 30 to the waistband 4 of the user's garment 2 instead of the lower leg portion of the garment. Elongated attachment portion may be attached to the waistband by buttons, snaps, hook-and-loop fasteners or any other fastener that is known in the art. Elongated attachment portion 32 may be attached to fluid barrier layer 20 of incontinence device 30 by any means known in the art such as, for example, by sewing. Elongated attachment portion 32 may also be attached to absorption layer 18 and/or top-sheet layer 28.

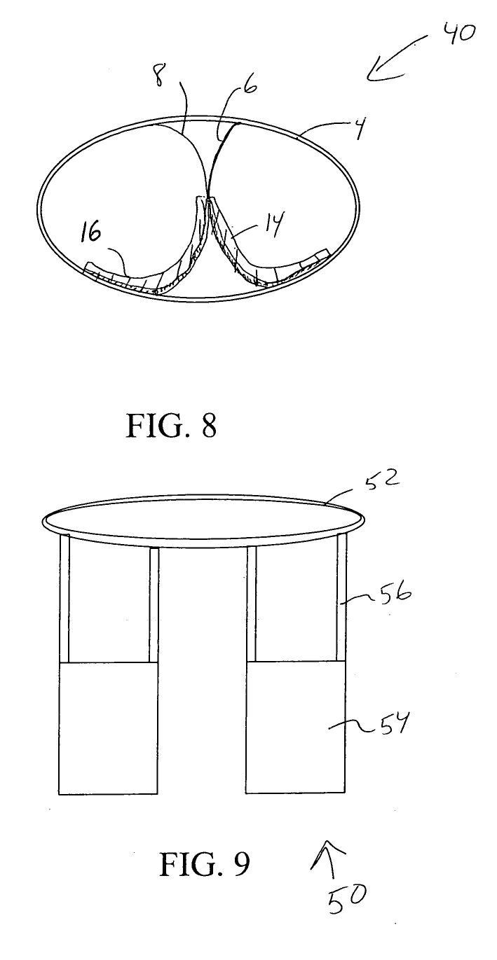

FIG. 8 illustrates another embodiment of an incontinence device 40 made in accordance with the present invention. In this embodiment, absorber flaps 14 and 16 are positioned to cover an inner portion of the user's thigh as well as at least a portion of the front of the user's thigh. Incontinence device 40 may be the same or similar to any of the incontinence devices described herein with reference to FIGS. 1 to 7.

FIG. 9 illustrates another embodiment of an incontinence device 50 made in accordance with the present invention. Incontinence device 50 includes an elastic waistband 52 for encircling the user's waist and at least one absorber flap 54, in this case two absorber flaps are illustrated. Absorber flap 54 includes an elongated attachment portion 56 for attaching absorber flap 54 to waistband 52. Absorber flap 54 may be the same or similar to the absorber flaps described with reference to FIGS. 1 to 7. incontinence device 50 may be worn by the user with or without an under garment.

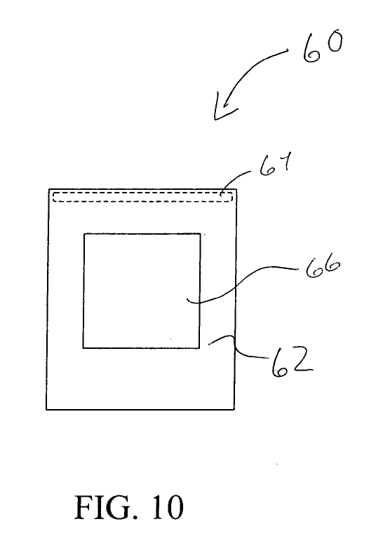

FIG. 10 illustrates an embodiment of an absorber flap 60 made in accordance with the present invention. Absorber flap 60 includes a liquid barrier layer and an absorption layer 62 as described above with relation to FIGS. 1 to 4. Absorber flap 60 may be attached to the garment via attachment portion 64 by any means known in the art. Absorber flap 60 also includes a pocket 66 disposed adjacent absorption layer 62. Pocket 66 may be formed integrally with absorption layer 62 and may be composed of the same or similar material. In another embodiment, pocket 66 is composed of the same or similar material as described above for top-sheet layer 28 illustrated in FIG. 7. In use, the user places at least a portion of his penis within pocket 66 so that any urine that may leak out the tip of the penis will be immediately absorbed by absorption layer 62. The placement and size of the pocket 66 may be adjusted to suit a particular need.

While the embodiments of the present invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. An incontinence undergarment device, comprising:
at least one absorber flap, the absorber flap including an absorption layer; a liquid barrier layer adjacent the absorption layer, and an attachment portion positioned at an upper region of the flap to attach to a front leg portion of an undergarment.

2. The device of claim 1 wherein the absorption layer comprises a material chosen from the group consisting of cotton, super absorbent polymer, paper wadding and cellulose fiber

3. The device of claim 1 wherein the liquid barrier layer comprises a nonporous material.

4. The device of claim 3 wherein the nonporous material comprises a polyethylene film.

5. The device of claim 1 further comprising a top sheet layer.

6. The device of claim 5 wherein the top sheet layer comprises a porous hydrophobic material.

7. The device of claim 6 wherein the porous hydrophobic material comprises polyester or polypropylene non-woven fabric.

8. The device of claim 1 wherein the absorption layer includes a pocket disposed adjacent a user's skin.

9. The device of claim 1 wherein the attachment portion comprises an elongated attachment portion for attaching to an elastic band for extending around a waist of a user.

10. The device of claim 9 wherein the elastic band is integral with an undergarment.

11. An incontinence undergarment, comprising:
a boxer shorts;
a pair of absorber flaps, each absorber flap including an absorption layer; a liquid barrier layer adjacent the absorption layer, and an attachment portion positioned at an upper region of the flap to attach to a front leg portion of the boxer shorts, wherein a portion of the absorber flaps hangs below the front leg portions when a user is standing.

12. The undergarment of claim 11 wherein the absorption layer comprises a material chosen from the group consisting of cotton, super absorbent polymer, paper wadding and cellulose fiber.

13. The undergarment of claim 11 wherein the liquid barrier layer comprises a nonporous material.

14. The undergarment of claim 13 wherein the nonporous material comprises a polyethylene film.

15. The undergarment of claim 11 further comprising a top sheet layer.

16. The undergarment of claim 15 wherein the top sheet layer comprises a porous hydrophobic material.

17. The undergarment of claim 16 wherein the porous hydrophobic material comprises polyester or polypropylene non-woven fabric.

18. An incontinence undergarment, comprising:
a boxer shorts; and
flap means for absorbing urine, the flap means attached to a leg portion of the boxer shorts.

19 The undergarment of claim 17 further comprising an attachment means for attaching the flap means to the leg portion of the boxer shorts.
